(19) United States
(12) Patent Application Publication
(10) Pub. No.: US 2005/0124949 A1
(43) Pub. Date: Jun. 9, 2005

(54) Unitized cover and transfer layer and process for making the same

(76) Inventors: John Steffen, Sugar Hill, GA (US); David Bye, Lawrenceville, GA (US); Lisa Baker, Lexington, NC (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/896,789

(22) Filed: Jul. 22, 2004

Related U.S. Application Data
(60) Provisional application No. 60/489,141, filed on Jul. 22, 2003.

(51) Int. Cl. B32B 7/02; A61F 13/15

(52) U.S. Cl. 604/367

(57) ABSTRACT

The present invention is directed to an unitized cover and transfer layer for hygiene applications that have the appearance, strength, and drape of a nonwoven and the surface characteristics of a film. The lightweight apertured film surface the present invention is comprised of a series of void spaces that act to collect liquids and channel such liquid into an associated underlying absorbent layer, while reducing potential release of particulates inherent to the absorbent layer. Further, the unitized cover and transfer layer of the present invention exhibits significantly reduced frictional noise and discomfort induced by prolonged contact.

### TECHNICAL FIELD

The present invention generally relates to an apertured film laminate, and more specifically to an apertured film and nonwoven laminate wherein the laminate has the appearance, strength, and drape of a nonwoven and the surface characteristics of a film, said laminate structure especially suited as a unitized cover and transfer layer for absorbent articles.

### BACKGROUND OF THE INVENTION

Films are used in a wide variety of applications where the engineered qualities of the film can be advantageously employed as a component substrate. The use of selected thermoplastic polymers in the construction of film products, selected treatment of the polymeric films (either while in melt form or in an unitized structure), and selected use of various mechanisms by which the film is unitized into a useful construct, are typical variables by which to adjust and alter the performance of the resultant polymeric film product.

The formation of finite thickness films from thermoplastic polymers is a well known practice. Thermoplastic polymer films can be formed by either dispersion of a quantity of molten polymer into a mold having the dimensions of the desired end product, known as a thermo-formed or injection-molded film, or by continuously forcing the molten polymer through a die, known as an extruded film. Extruded thermoplastic polymer films can either be formed such that the film is cooled then wound as a completed product, or dispensed directly onto a substrate material to form a composite material having performance of both the substrate and the film layers. Examples of suitable substrate materials include other films, polymeric or metallic sheet stock and woven or nonwoven fabric.

To further improve the performance of the thermoplastic polymer film when used in composite material manufacture, various additives are admixed with the thermoplastic polymer prior to or during extrusion. Typical additives employed are those selected from various colorants or opacifiers, such as titanium dioxide. If there is a desire to form a composite wherein the thermoplastic polymer film will be exposed to a transitory temperature above the melting temperature of the polymer, antioxidants can be incorporated into the mix to aid in reducing thermal degradation. In the event where the family of thermoplastic polymers to be used in the extruded film exhibits a dissimilar characteristic such as surface energy from the thermoplastic polymer of the substrate material, compatibilizers are incorporated into the polymer mix.

The utilization of a film-nonwoven laminate construct in various end-use applications, such as surgical drapes, and hygiene applications, such as sanitary napkins, is known to those skilled in the art. The prior art to the aforementioned applications include U.S. Pat. No. 4,033,341 to Scrivens and U.S. Pat. No. 4,184,498 to Franco, which are both hereby incorporated by reference. Hygiene applications, specifically feminine care products, such as sanitary napkins and panty liners, diaper, and incontinence pads, and the like, tend to use either a film or nonwoven cover sheets, with both substrates posing advantages and disadvantages. Whereas some end-users prefer nonwoven covers for the softness associated with the fabric, the nonwoven cover often lacks the feeling of dryness desired in fem-care products. On the other hand, some end-users prefer film covers due to the dry feeling offered by the film; however the film covers often tend to feel hot as well, and doesn't offer the same level of comfort as a nonwoven.

A need remains for a lightweight film laminate that can be utilized as a unitized cover and transfer layer in fem-care products and other absorbent articles, so as to provide a combination of beneficial nonwoven and film characteristics suitable for both groups of end-users.

### SUMMARY OF THE INVENTION

The present invention is directed to an unitized cover and transfer layer for hygiene applications that have the appearance, strength, and drape of a nonwoven and the surface characteristics of a film. The lightweight apertured film surface the present invention is comprised of a series of void spaces that act to collect liquids and channel such liquid into an associated underlying absorbent layer, while reducing potential release of particulates inherent to the absorbent layer. Further, the unitized cover and transfer layer of the present invention exhibits significantly reduced frictional noise and discomfort induced by prolonged contact.

Traditionally, the apertured film process as is disclosed in U.S. Pat. No. 4,690,679, to Mattingly III, et al., has produced 25 to 27 gsm film at the lightest weight utilizing an ethyl vinyl acrylate/linear low density polyethylene chemistry. The present invention incorporates a light (7 to 15 gsm) fibrous or filamentary substrate, which allows for a lightweight extruded coating of a 20 gsm film.

In accordance with the present invention, the lightweight unitized cover and transfer layer construct is formed by extruding a thin film on the order of 20 gsm onto a lightweight fibrous or filamentary substrate. Subsequent to coating the substrate with the film, the laminate is embossed, using micro-embossing, macro-embossing, or both, wherein during the embossing process the film is forced into the fibrous or filamentary network that make up the land areas of the embossing pattern. The molten polymer is extruded at the heated roll, comes in contact with the substrate at the embossed roll, and then advances onto the cooled roll. The resulting fabric has improved air permeability due to more uniform, clear aperture formations, improved caliper, and drapeability.

The unitized cover and transfer layer of the present invention is lightweight, wherein the film layer is on the order of 20 gsm and the substrate layer 7-15 gsm. For hygienic absorbent article applications, the apertured film side is the body facing layer, channeling exudates through the apertures and into the nonwoven transfer layer to be sequestered into the absorbent core.

### BRIEF DESCRIPTION OF THE DRAWINGS

FIG. 1 schematic representation of the processing apparatus for producing an apertured film in accordance with the principles of the present invention;

FIG. 2 is a depiction of a preferred method of making the unitized cover and transfer layer of the present invention;

FIG. 3 is a photomicrograph of the unitized cover and transfer layer of the present invention:

FIG. 4 is a magnified photomicrograph of FIG. 3; and

FIG. 5 is a plan view of a diaper in an uncontracted state.

### DETAILED DESCRIPTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

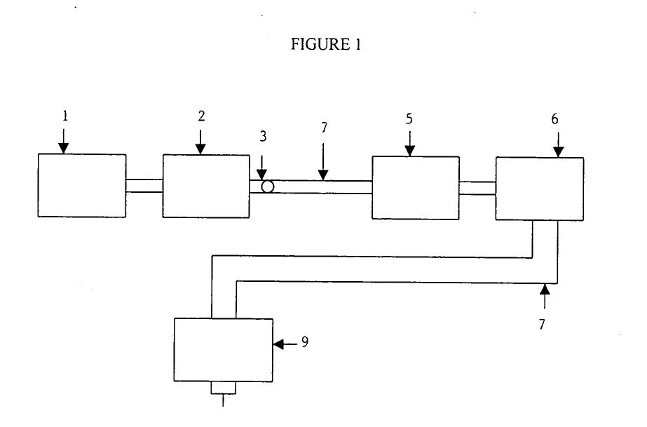

FIG. 1 depicts a representative direct extrusion film process. Blending and dosing system 1, comprising at least two hopper loaders for polymer chip and a mixing hopper. Variable speed augers within both hopper loaders transfer predetermined amounts of polymer chip and additive pellet to the mixing hopper. The mixing hopper contains a mixing propeller to further the homogeneity of the mixture. Basic volumetric systems such as that described, are a minimum requirement for the blending zone system.

The polymer chip and additive pellet blend feeds into a multi-zone extruder 2 as supplied by the Wellex Corporation. In this particular system, a five zone extruder was employed with a 2 inch water-jacketed bore and a length to diameter ratio of 24 to 1.

Upon mixing and extrusion from multi-zone extruder 2, the polymer compound is conveyed via heated polymer piping 7 through screen changer 3, wherein breaker plates having different screen meshes are employed to retain solid or semi-molten polymer chips and other macroscopic debris. The mixed polymer is then fed into melt pump 5.

Melt pump 5 operates in dynamic feed back with the multi-zone extruder 2 to maintain the desired pressure levels. A gear-type melt pump was employed to respond to pressure levels by altering the speed of the extruder to compensate for deviations from the pressure set point window.

The metered and mixed polymer compound then enters combining block 6. The combining block allows for multiple film layers to be extruded, the film layers being of either the same composition or fed from different systems as described above. The combining block 6 is directed into die body 9 by additional heated polymer piping 7.

The particular die body 9 employed in this system is a 37 inch wide EDI Automatic Die with die bolt control as supplied by EDI. The die body 9 is positioned in an overhead orientation such that molten film extrusion 15 is deposited at the heated roll 14, wherein the molten film coats the substrate at the embossing roll 10 and the laminate advance onto the cooling roll 11.

The film substrate of the present invention may be that of various olefinic polymers including, but are not limited to, isotactic polypropylene, linear low-density polyethylene, low-density polyethylene, high-density polyethylene, medium low-density polyethylene, very low-density polyethylene, amorphous polypropylene, polyethylene copolymers, polypropylene copolymers, polybutylene, ethylene/vinyl acetate copolymer, ethylene/ethyl acrylate copolymer, ethylene/methyl acrylate copolymer, polystyrene, plastomers, and the combination thereof.

It is within the purview of the present invention that the substrate to be coated with the molten polymer be either of fibrous or filamentary formation. Staple fibers used to form nonwoven fabrics begin in a bundled form as a bale of compressed fibers. In order to decompress the fibers, and render the fibers suitable for integration into a nonwoven fabric, the bale is bulk-fed into a number of fiber openers, such as a garnet, then into a card. The card further frees the fibers by the use of co-rotational and counter-rotational wire combs, then depositing the fibers into a lofty batt. The lofty batt of staple fibers can then optionally be subjected to fiber reorientation, such as by air-randomization and/or cross-lapping, depending upon the ultimate tensile properties of the resulting nonwoven fabric. The fibrous batt is unitized into a nonwoven fabric by application of suitable bonding means, including, but not limited to, use of adhesive binders, thermo-bonding by calender or through-air oven, and hydroentanglement. Preferably, the substrate of the unitized cover and transfer layer has a basis weight range of about 7 to 30 grams per square meter, more preferably 8 to 20 grams per square meter, and most preferably a basis weight range of about 10 to 15 grams per square meter. Further, the substrate may be filamentary, such as a spunbond or meltblown web. The fibers or filaments of the spunmelt can be selected from a group of polyesters, polyamides, or polyolefins, such as polypropylene, polyethylene, and the combinations thereof. The fibers or filaments may also be one of a multi-component configuration of the above mentioned polymers and preferably has a basis weight between 7 and 15 gsm.

A spunbond process involves supplying a molten polymer, which is then extruded under pressure through a large number of orifices in a plate known as a spinneret or die. The resulting continuous filaments are quenched and drawn by any of a number of methods, such as slot draw systems, attenuator guns, or Godet rolls. The continuous filaments are collected as a loose web upon a moving foraminous surface, such as a wire mesh conveyor belt. When more than one spinneret is used in line for the purpose of forming a multi-layered fabric, the subsequent webs is collected upon the uppermost surface of the previously formed web. The web is then at least temporarily consolidated, usually by means involving heat and pressure, such as by thermal point bonding. Using this bonding means, the web or layers of webs are passed between two hot metal rolls, one of which has an embossed pattern to impart and achieve the desired degree of point bonding, usually on the order of 10 to 40 percent of the overall surface area being so bonded.

A related means to the spunbond process for forming a layer of a nonwoven fabric is the melt blown process. Again, a molten polymer is extruded under pressure through orifices in a spinneret or die. High velocity air impinges upon and entrains the filaments as they exit the die. The energy of this step is such that the formed filaments are greatly reduced in diameter and are fractured so that microfibers of finite length are produced. Additionally, nano-fibers of infinite length, wherein the average fiber diameter of the nano-fiber is in the range of less than or equal to 1000 nanometers, and preferably less than or equal to 500 nanometers may be incorporated. The extruded multiple and continuous filaments can be optionally imparted with a selected level of crimp, then cut into fibers of finite staple length.

These thermoplastic resin staple fibers can then be subsequently used to form textile yarns or carded and unitized into nonwoven fabrics by appropriate means, as exemplified by thermo-bonding, adhesive bonding, and hydroentanglement technologies. The process to form either a single layer or a multiple-layer fabric is continuous, that is, the process steps are uninterrupted from extrusion of the filaments to form the first layer until the bonded web is wound into a roll.

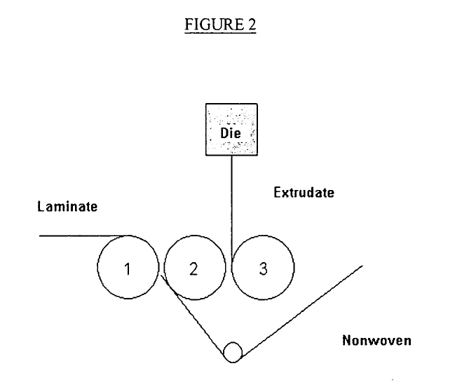

In a preferred embodiment and as depicted in FIG. 2, the olefinic molten polymer is extruded at the heated roll (3), comes in contact with the substrate at the embossed roll (2), and then advances onto the cooled roll (1). The resulting unitized cover and transfer layer has improved air permeability due to more uniform, clear aperture formations, improved caliper, and drapeability. Subsequent to coating the substrate with the film, the laminate is embossed, using micro-embossing, macro-embossing, or both, wherein during the embossing process the film is forced into the fibrous or filamentary network that make up the land areas of the embossing pattern.

It is further within the purview of the present invention that the film laminate optionally comprise an aesthetic or performance-modifying additive, wherein the modifying additive may be incorporated into the molten polymer or topically applied to the unitized cover and transfer layer. The use of such additives may include, but are not limited to, wetting agents, pigments, anti-microbials, emollients, fragrances, and the combination thereof.

### EXAMPLE

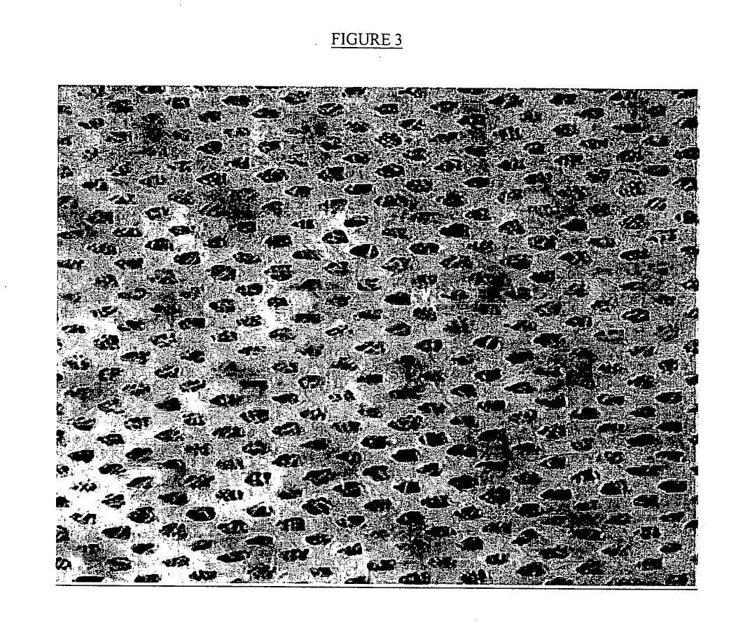

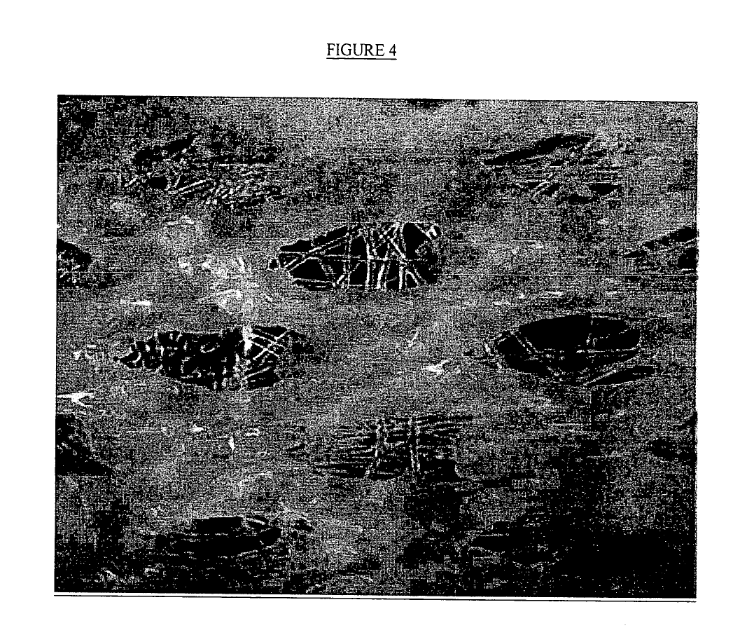

In accordance with the present invention, a 10 gsm SBPP was coated with 20 gsm of Comfort Silk® film, which is made commercially available by Polymer Group, Inc. Table 1 contains the physical properties of the resultant structure. The resultant structure has uniform and clear aperture formations, with a 332 cfm Frasier air permeability. Further, the machine direction handleometer reading averaged 12.5 grams. Further still, the 20 gsm film/10 gsm nonwoven structure possessed an embossed thickness of 315 microns. The composite structure has the appearance, strength and drape of a nonwoven and the surface characteristics of a film. The adhesion mechanism is primarily fiber penetration into the film layer caused by macro-embossing. Optionally, the entire laminate or select regions may be micro-embossed in addition to the macro-embossing. FIGS. 3 and 4 are representative of the a unitized cover and transfer layer of the present invention.

Various hygiene, industrial, and medical end-use articles may benefit from the lightweight laminate, wherein the laminate of the invention is especially suited for a unitized cover and transfer layer for hygienic products, such as sanitary napkins, including panty liners, as well as other absorbent articles. A general construct for an absorbent article includes a cover and a backsheet affixed about a central absorbent core. Representative prior art to such article fabrication include U.S. Pat. No. 4,029,101, No. 4,184,498, No. 4,195,634, No. 4,408,357 and No. 4,886,513, which are incorporated herein by reference.

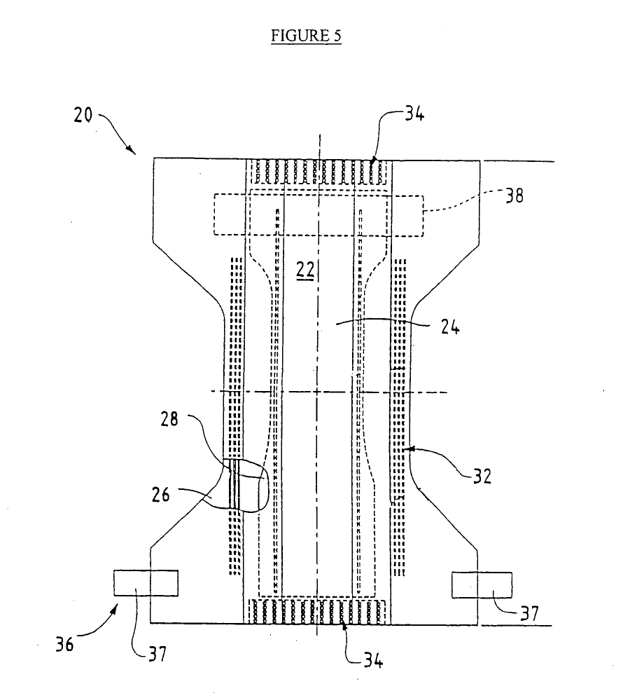

An absorbent article incorporating the unitized cover and transfer layer of the present invention is represented by the unitary disposable absorbent article, diaper 20, shown in FIG. 5. As used herein, the term “diaper” refers to an absorbent article generally worn by infants and incontinent persons that is worn about the lower torso of the wearer. It should be understood, however, that the present invention is also applicable to other absorbent articles such as incontinence briefs, incontinence undergarments, diaper holders and liners, feminine hygiene garments, training pants, pull-on garments, and the like.

FIG. 5 is a plan view of a diaper 20 in an uncontracted state (i.e., with elastic induced contraction pulled out) with portions of the structure being cut-away to more clearly show the construction of the diaper 20. As shown in FIG. 5, the diaper 20 preferably comprises a containment assembly 22 comprising a liquid pervious topsheet 24; a liquid impervious backsheet 26 joined to the topsheet; and an absorbent core 28 positioned between the topsheet 24 and the backsheet 26. The absorbent core 28 has a pair of opposing longitudinal edges, an inner surface and an outer surface. The diaper can further comprise elastic leg features 32; elastic waist features 34; and a fastening system 36, which preferably comprises a pair of securement members 37 and a landing member 38.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

TABLE 1

| | Film | | Total | Gloss | Smoothness | Thickenss | Air Perm | MD Hand | | |
|---|---|---|---|---|---|---|---|---|---|---|
| Nonwoven | Weight | Substrate | Weight | units | units | microns | cfm | grams | Strikethrough | Rewet |
| 10 gsm SBPP | 20 | Phobic | 30 | 6.3 | 4.5 | 314 | 332 | 13 | 3.58 | 2.85 |
| 17 gsm SBPP | 15 | Phobic | 32 | 5.2 | 1.5 | 323 | 267 | 16 | 3.31 | 1.88 |
| 10 gsm SBPP | 15 | Philic | 25 | 5 | 1.4 | 340 | 401 | 11 | 1.4 | 2.44 |
| 17 gsm SBPP | 20 | Philic | 37 | 4.9 | 1.1 | 320 | 236 | 21 | 1.49 | 2.89 |

1. A lightweight unitized cover and transfer layer construct for use in an absorbent article, wherein said construct comprises a layer of polyolefinic apertured film having a basis weight equal to or less than 20 gsm and a fibrous or filamentary web layer in a range between 7-30 grams per square meter.

2. A lightweight unitized cover and transfer layer construct as in claim 1, wherein said polyolefinic film is selected from the group consisting of isotactic polypropylene, linear low-density polyethylene, low-density polyethylene, high-density polyethylene, medium low-density polyethylene, very low-density polyethylene amorphous polypropylene, polyethylene copolymers, polypropylene copolymers, polybutylene, ethylene/vinyl acetate copolymer, ethylene/ethyl acrylate copolymer, ethylene/methyl acrylate copolymer, polystyrene, plastomers, and the combination thereof.

3. A sanitary napkin comprising a lightweight unitized cover and transfer layer construct, wherein said construct comprises a layer of polyolefinic apertured film having a basis weight equal to or less than 20 gsm and a fibrous or filamentary web layer in a range between 7-30 grams per square meter, an absorbent core, and a liquid impermeable backsheet.

4. A diaper comprising a lightweight unitized cover and transfer layer construct, wherein said construct comprises a layer of polyolefinic apertured film having a basis weight equal to or less than 20 gsm and a fibrous or filamentary web layer in a range between 7-30 grams per square meter, an absorbent core, and a liquid impermeable backsheet.
